(19) United States
(12) Patent Application Publication
(10) Pub. No.: US 2011/0274667 A1
(43) Pub. Date: Nov. 10, 2011

(54) METHODS FOR OBTAINING CELL POPULATIONS FROM ADIPOSE TISSUE

(75) Inventors: Anthony Donofrio, Westmont, IL (US); Delara Motlagh, Barrington, IL (US); David L. Amrani, Glendale, WI (US); Amy Cohen, Grayslake, IL (US)

(73) Assignees: Baxter Healthcare S.A., Glattpark (Opfikon) (CH); Baxter International Inc., Deerfield (IL)

(21) Appl. No.: 13/124,632

(22) PCT Filed: Oct. 19, 2009
(86) PCT No.: PCT/US2009/061185

§ 371 Date: Jul. 20, 2011

Related U.S. Application Data
(60) Provisional application No. 61/106,353, filed on Oct. 17, 2008.

(51) Int. Cl. A61K 35/12; A61P 9/10; A61P 13/12; A61P 37/06; A61P 27/02; A61P 17/02; C12N 5/077; A61P 3/10

(52) U.S. Cl. 424/93.7; 435/325

(57) ABSTRACT

Provided herein are methods of efficiently obtaining large numbers of viable, freshly isolated cells from small amounts of adipose tissue, as well as methods of enriching or selecting for target cell populations found therein. In certain embodiments, the method of obtaining a population of cells from adipose tissue comprises incubating the adipose tissue in a solution comprising an enzyme at a concentration which is at least 200 U/ml solution and not more than about 319 U/ml solution. In some embodiments, the method is devoid of any steps which expand the population of cells obtained. In certain aspects, the method further comprises positive or negative selection steps for obtaining an enriched population of target cells from adipose tissue. Related methods of preparing a pharmaceutical composition comprising cells for administration to a patient and methods of treating a disease or medical condition in a patient are further provided herein.

### CROSS REFERENCE TO RELATED APPLICATIONS

This application claims priority to U.S. Provisional Patent Application No. 61/106,353, filed on Oct. 17, 2008, which is incorporated by reference in its entirety.

### BACKGROUND

It has been a goal of scientists and doctors to use stem cells to treat diseases by administering these cells to sites of disease, where it is hoped that the cells will regenerate or repair the tissue. While mesenchymal stem cells (also called stromal stem cells) exhibit extensive proliferative capacity and the ability to generate progeny of the connective tissue lineages (bone, cartilage, tendon, fat, etc.), these cells can be difficult to harvest in numbers suitable for clinical use.

Human adipose tissue has been shown to contain a population of cells that has extensive proliferative capacity, as well as the ability to differentiate into multiple cell lineages. These cells, referred to as adipose tissue-derived stem cells (ADSCs) or adipose stromal stem cells (ASCs), are generally similar, though not identical, to mesenchymal stem cells (also referred to as marrow stromal cells). Fraser et al., Methods in Molec Biol 449: 59-67 (2008).

While methods of obtaining cells, e.g., ADSCs, from adipose tissue are known in the art, these methods remain to be optimized for efficiency, such that the maximal number of viable cells are obtained from a small amount of adipose tissue. For example, Fraser et al., 2008, supra, discloses obtaining cells from adipose tissue, in which only 2×10⁴ cells are isolated from the tissue, whereas typically about 10⁷-10⁹ cells are needed for infusion into a patient for therapeutic purposes (International Patent Application Publication No. WO 03/080801). Also many of these methods involve plating or tissue culture steps of the cells obtained from adipose tissue for purposes of cell proliferation and expansion. Because the phenotypes of cells change as a function of time in tissue culture, methods of obtaining a large number of cells with little to no in vitro tissue culture steps are needed.

Thus, there is a need in the art for optimized methods which efficiently obtain large numbers of viable, freshly isolated cells from small amounts of adipose tissue. Methods for enriching the cell product from adipose tissue for cells of interest, e.g., CD34-positive cells, are further needed.

### BRIEF SUMMARY OF THE INVENTION

Provided herein are methods of efficiently obtaining large numbers of viable, freshly isolated cells from small amounts of adipose tissue, as well as methods of enriching or selecting for target cell populations found therein.

In certain embodiments, the method of obtaining a population of cells from adipose tissue comprises incubating the adipose tissue in a solution comprising an enzyme at a concentration which is at least 200 U/ml solution and not more than about 319 U/ml solution. In some embodiments, the method is devoid of any steps which expand the population of cells obtained. In certain aspects, the method further comprises positive or negative selection steps for obtaining an enriched population of target cells from adipose tissue. In this regard, the invention also provides a method of obtaining an enriched population of target cells from adipose tissue, comprising obtaining a population of cells from adipose tissue and incubating the population of cells in a second solution comprising a primary antibody that separates the population of cells into a subpopulation comprising target cells and a subpopulation substantially devoid of target cells, thereby obtaining an enriched population of target cells.

The invention further provides the population of cells (e.g., the enriched population of target cells) obtained in accordance with the methods described herein.

Related methods of preparing a pharmaceutical composition comprising the population of cells (e.g., the enriched population of target cells) obtained from the adipose tissue, as well as the pharmaceutical compositions prepared in accordance with these methods, and methods of using the pharmaceutical composition in methods of treating a disease or medical condition in a patient, are provided herein.

### BRIEF DESCRIPTION OF THE DRAWINGS

FIG. 1 represents a schematic diagram of an antibody-based positive selection technique, wherein the target cells (or selected cells marked with an “S”) are CD34-positive cells, which are part of the stromal vascular fraction (SVF) of adipose tissue. The primary antibody (anti-CD34 mAb) binds to the CD34-positive cells (marked with “S”) and a secondary antibody (SAM Ig antibody) in turn binds to the primary antibody. The secondary antibody is bound to a paramagnetic bead and a magnet is used to remove the immune complexes from the SVF fraction. To release the CD34-positive cells, a release peptide (shown as a triangle) is added to the complexes and displacement of the primary antibody and the CD34 antigen on the target cells occurs to provide a population of purified CD34-positive cells.

FIG. 2 represents a multi-color flow cytometric panel of cells obtained after adipose tissue digestion as described herein. Bright and dim CD34 ASCs and MSCs, as well as lymphocytes, endothelial cells and hematopoietic progenitors are shown. Debris or dead cells are also a gated population.

FIG. 3 represent flow cytometric panels of SVF before (left panel) and after selection (right panel) as outlined in FIG. 1. CD34 bright/CD45−, CD34 bright/CD45+, CD34 dim/CD45− populations are shown.

FIG. 4 represents flow cytometric data which is the same as FIG. 2, except that Populations A-C are labeled.

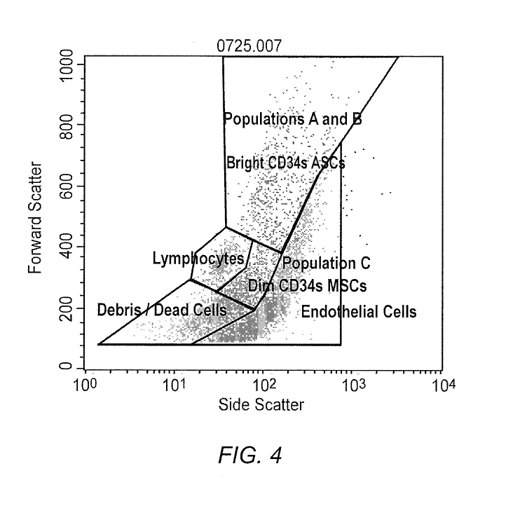

FIG. 5 represents a graph of a Laser Doppler Imaging Time Course. The data are expressed as a percent perfusion in the ischemic limb compared to the non-ischemic limb of mice administered PBS only, unselected cells, or CD34-positive selected cells as described herein.

### DETAILED DESCRIPTION OF THE INVENTION

The invention provides methods of obtaining a population of cells from adipose tissue. The methods comprise incubating the adipose tissue in a solution comprising an enzyme at a concentration which is optimized to achieve release of the greatest number of cells per ml of adipose tissue without sacrificing cell viability.

Adipose Tissue

With regards to the invention, the adipose tissue may be any body fat (or fat) which is loose connective tissue composed of adipocytes. The adipose tissue in some embodiments is bone marrow adipose tissue, brown adipose tissue, mammary adipose tissue, mechanical adipose tissue, or white adipose tissue. The adipose tissue may be obtained from any part of the body of the donor. In certain embodiments the adipose tissue is obtained from the thigh, buttocks, abdomen, or arms of the donor. In other embodiments, the adipose tissue is obtained from the breast, neck, back, or calves of the donor. In yet other embodiments, the adipose tissue is obtained from the heart, kidney, aorta, gonads, retroorbital or palmar fat pads, In certain aspects, the adipose tissue is subcutaneous fat. The adipose tissue donor may be any host, including but not limited to any of those described herein. In some embodiments, the donor is a mammal. In specific embodiments, the donor is a human.

The adipose tissue may be obtained from the donor through any suitable method for obtaining adipose tissue. Such methods are known in the art. In some embodiments, the adipose tissue is obtained from the donor through surgery or by liposuction. Accordingly, the adipose tissue in some embodiments is a lipoaspirate.

With regards to the invention, the amount of adipose tissue incubated with the enzyme solution can be any amount, e.g., 0.01, 0.1, or 1 g to 5, 10, 50, or 100 g of adipose tissue.

Enzyme Solutions

With regards to the invention, the term “solution” as used herein refers to any medium suitable for contacting tissue or cells with an enzyme. The solution can have any viscosity or consistency and in some aspects, the solution is an aqueous solution. In alternative embodiments, the solution is a semi-solid medium at room temperature. In certain specific aspects, the solution is a tissue culture media as further described herein.

The enzyme contained in the solution can be any enzyme known for digesting tissue, e.g., connective tissue (e.g., connective tissue found in adipose tissue). In some aspects, the enzyme is a connective tissue digesting enzyme. In some embodiments, the enzyme is obtained or derived from an animal or non-animal source. In some embodiments, the enzyme is a protease, a peptidase, or a proteinase. In certain specific embodiments, the enzyme is a collagenase, trypsin, or dispase, or a functionally equivalent, variant, derivative, mutant, or analog thereof. The enzyme in some embodiments is a mixture of enzymes, e.g., a mixture comprising at least one or two of collagenase, trypsin, or dispase. The enzyme in certain embodiments is Liberase Blendzyme (Roche). Suitable enzymes for purposes herein are known in the art and are commercially available from companies, including, but not limited to Sigma Aldrich (St. Louis, Mo.), Worthington Biochemcial (Lakewood, N.J.), and Roche (Indianapolis, Ind.).

Enzyme Concentration

The enzyme concentration should be great enough to achieve digestion of the adipose tissue for release of the greatest amount of cells from the tissue. Accordingly, in some embodiments, the method comprises incubating the adipose tissue in a solution comprising an enzyme at a concentration which is at least about 200 U/ml solution (e.g., at least about 205 U/ml solution, at least about 210 U/ml solution, at least about 215 U/ml solution, at least about 220 U/ml solution, at least about 225 U/ml solution, at least about 230 U/ml solution, at least about 235 U/ml solution, at least about 240 U/ml solution, at least about 245 U/ml solution, at least about 250 U/ml solution, at least about 260 U/ml solution, at least about 265 U/ml solution, at least about 270 U/ml solution, at least about 280 U/ml solution, at least about 285 U/ml solution, at least about 290 U/ml solution, at least about 300 U/ml solution, at least about 310 U/ml solution, at least about 315 U/ml solution or more).

With regards to the invention, care should be taken not to overdigest the adipose tissue such that the cells are no longer viable. Those of skill in the art will appreciate that overdigestion is avoided by balancing the amount of enzyme in the digestion reaction with the time of the digestion reaction (as discussed below). Accordingly, in some embodiments, incubating the adipose tissue in a solution comprising an enzyme at a concentration which is not more than about 500 U/ml solution (e.g., not more than about 475 U/ml solution, not more than about 450 U/ml solution, not more than about 425 U/ml solution, not more than about 400 U/ml solution, not more than about 375 U/ml solution, not more than about 350 U/ml solution, not more than about 325 U/ml solution, not more than about 320 U/ml solution, not more than about 319 U/ml solution, not more than about 315 U/ml solution, not more than about 310 U/ml solution, not more than about 305 U/ml solution).

In certain embodiments, the method comprises incubating the adipose tissue in a solution comprising an enzyme at a concentration which is between about 190 U/ml and 319 U/ml (e.g., between about 200 U/ml and about 300 U/ml, between about 205 U/ml and 295 U/ml, about 210 U/ml and 290 U/ml, between about 215 U/ml and about 285 U/ml, between about 220 U/ml and about 280 U/ml, between about 225 U/ml and about 275 U/ml, between about 230 U/ml and about 270 U/ml, between about 235 U/ml and about 265 U/ml, between about 240 U/ml and about 260 U/ml, between about 245 U/ml and about 255 U/ml.

In certain embodiments, the method comprises incubating the adipose tissue in a solution comprising an enzyme at a concentration which is about 245 U/ml, about 246 U/ml, about 247 U/ml, about 248 U/ml, about 249 U/ml, about 250 U/ml, about 251 U/ml, about 252 U/ml, about 253 U/ml, about 254 U/ml, or about 255 U/ml).

In some embodiments, the ratio of the amount (e.g., volume) of adipose tissue to the amount of enzyme (volume of enzyme solution) is about 1:1, 1:2, 2:1, 1:3, 3:1, 1:4, 4:1, 1:5, 5:1.

Digestion Times

In some embodiments, incubation of the adipose tissue with the solution containing the enzyme occurs for a time between about 5 minutes and 5 hours or more. In some specific embodiments, incubation of the adipose tissue with the enzyme solution occurs for a time between about 15 minutes and about 1.5 hours, between about 25 minutes and about 1.25 hours, or between about 30 minutes and about 60 minutes. In some aspects, the adipose tissue is incubated with the enzyme solution for a time between about 45 and 55 minutes. In this regard, the adipose tissue in certain embodiments is incubated in the enzyme solution for about 45, about 46, about 47, about 48, about 49, about 50, about 51, about 52, about 53, about 54, or about 55 minutes.

Again, those of skill in the art will appreciate that overdigestion is avoided by balancing the amount of enzyme in the digestion reaction (as discussed above) with the time of the digestion reaction.

Other Digestion Conditions and Steps

In some embodiments, the temperature at which the adipose tissue is incubated in the enzyme solution is any suitable temperature which permits digestion (e.g., which does not inactivate the enzyme). The temperature in some embodiments, is between about 20 degrees C. and 50 degrees C. (e.g., about 25° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C.).

The incubation of adipose tissue with the enzyme solution in some embodiments is performed in the presence of shaking, vibrating, rotating, or other movement or mechanical agitation of the container holding the adipose tissue and enzyme solution. In other embodiments, the incubation is maintained without any of the foregoing.

In some embodiments, the methods comprise further steps, such as any of the tissue harvesting, tissue processing, and tissue washing steps described in the art. See for example, WO 03/080801 and Fraser et al., 2008, supra. In some embodiments, the method comprises an inactivation step whereby the enzyme is inactivated. In some embodiments, a solution containing a high concentration of fetal bovine serum is used to inactivate the enzyme. In some embodiments, the method comprises separating the digested and undigested fractions of the adipose tissue. The method, in some embodiments, comprises centrifuging and/or filtering and/or washing and/or resuspending cell pellets.

Such additional considerations and further steps are either known in the art or are within the skill of the ordinarily skilled artisan to determine. See, for example, U.S. Pat. No. 6,777,231, International Patent Application Publication No. WO 03/080801, Fraser et al., 2008. supra, Bunnell et al., Methods 45: 115-120 (2008); Gimble et al., Cytotherapy 5:362:369 (2003); Locke et al., ANZ Journal of Surgery 79: 235-244 (2009); and Boquest et al., Methods in Molec Biol 325: 35-46 (2006).

In certain embodiments, the methods described herein obtain a large number of viable cells from adipose tissue without having to culture the cells obtained for purposes of cell proliferation or expansion. In this regard, in some embodiments, the method is devoid of any steps which expand the population of cells obtained from the adipose tissue. In other embodiments, the time in tissue culture is limited and minimal. In some embodiments, the cells are cultured or plated for no more than 8 hours, no more than 12 hours, no more than 18 hours, no more than 24 hours, no more than 36 hours, or no more than 48 hours.

Cell Population Obtained from Digested Adipose Tissue

The method of the invention is optimized for obtaining the greatest number of viable cells upon enzyme digestion of adipose tissue. In some embodiments, the number of viable cells obtained per ml of adipose tissue is at least about 10⁵, about 2×10⁵, about 3×10⁵, about 4×10⁵, about 5×10⁵, about 6×10⁵, about 7×10⁵, about 8×10⁵, about 9×10⁵, about 10⁶, about 10⁷ or more.

The cell population obtained from the adipose tissue in some embodiments is a heterogeneous cell population. In some embodiments, the cell population obtained from adipose tissue comprises adipose stromal stem cells, hematopoietic stem cells, mesenchymal stem cells, preadipocytes, endothelial cells or precursors thereof, fibroblasts, macrophages, lymphocytes, mastocytes, or a combination thereof. In some embodiments, the cell population is substantially free or minimally comprises adipocytes and red blood cells. The cell population is certain embodiments is the stromal vascular fraction of adipose tissue. Adipose derived stem cells are further described in U.S. Pat. No. 6,777,231. Human CD34+ stem cells are described in U.S. Pat. Nos. 5,130,144; 5,035,994; 4,965,204. In some embodiments, cell population obtained comprises adherent cells and/or non-adherent cells.

The cells of the population obtained from adipose tissue may be characterized by cell surface marker phenotype. In some embodiments, the population comprises cells that are positive for expression of cell markers such as any of those described herein In some embodiments, the population comprises cells that are positive for CD34. Further description of the cells obtained are described herein.

Selection/Enrichment of Cell Populations Obtained from Adipose Tissue

In some embodiments of the invention, the method further comprises selecting, isolating, enriching, or purifying for a subpopulation of desired or target cells. In this regard, the invention also provides a method of obtaining an enriched population of target cells from adipose tissue. Methods of purifying, cell sorting, and enriching for target cells are known in the art and include, for example, fluorescence activated cell sorting, centrifugation, and antibody-based capture techniques.

The method of obtaining an enriched population of target cells from adipose tissue in some embodiments, comprises obtaining a population of cells from adipose tissue in accordance with the methods described herein, e.g., incubating adipose tissue with a solution comprising a digestive enzyme, and incubating the population of cells with a primary antibody that separates the population of cells into a subpopulation comprising target cells and a subpopulation substantially devoid of target cells, thereby obtaining an enriched population of target cells.

As used herein, the term “substantially devoid” means lacking to some degree. It is recognized that “substantially devoid” is a relative term, and not to be necessarily construed as absolute or complete absence. Accordingly, in some aspects, the subpopulation which is substantially devoid of target cells comprises target cells, but only about 15% or less of the subpopulation are target cells. In some aspects, no more than about 10% of the subpopulation which is substantially devoid of target cells are targets cells. In some aspects, no more than about 9%, about 8%, about 7%, about 6%, about 5%, about 4%, about 3%, about 2%, or about 1% of the subpopulation which is substantially devoid of target cells are targets cells.

Negative Selection

The digested tissue may also be incubated with antibodies directed against cells of lesser or no interest to the final product. In some embodiments, when the target cells are CD34-positive cells, antibodies directed to the non-targeted cells may be incubated with the cells obtained following adipose tissue digestion or following incubation with paramagnetic beads coated with antibodies directed against the cell-specific antibodies. Through the process described above, such undesired (non-targeted) cells are then optionally isolated from the digest and removed therefrom. The resultant digest now contains lower to no concentration of the undesired cells and, consequently a higher concentration of the desired cells, for example, CD34+ cells. Examples of such cell removal include the reduction of the adipose digest of cells expressing CD45, Glycophorin-a and/or CD31.

In accordance with the foregoing, the subpopulation of target cells in some embodiments is enriched, selected, or purified by using a primary antibody which is an antibody specific for a cell marker which is not expressed or is expressed at low levels by the target cells. In certain specific embodiments, the primary antibody is an antibody specific for a cell marker selected from the group consisting of: CD45, Glycophorin A and CD31.

Positive Selection

The subpopulation of target cells in some embodiments is enriched, selected or purified by using a primary antibody which is an antibody specific for a cells marker which is expressed by the target cells. In some embodiments, the cell marker expressed by the target cells is CD34. In some embodiments, the target cells are CD34-positive cells. In specific embodiments, the primary antibody is an antibody which specifically binds to CD34. CD34 specific antibodies are known in the art and are commercially available. See, for example, U.S. Pat. No. 4,965,204. In some embodiments, the CD34 specific antibody is the antibody provided in an Isolex 300i kit (Baxter, Deerfield, Ill.).

In some aspects, the method of obtaining an enriched population of target cells from adipose tissue comprises both negative and positive selection procedures.

In some embodiments, the primary antibody is present with the population of cells at a final concentration within about 0.01 μg per 10⁶ target cells and about 10 μg per 10⁶ target cells, within about 0.1 μg per 10⁶ target cells and about 5 μg per 10⁶ target cells, or within about of about 1 μg per 10⁶ target cells to about 3 μg per 10⁶ target cells. In some embodiments, the primary antibody is at a final concentration of about 2.5 μg per 10⁶ target cells.

Beads

In some embodiments, the primary antibody which separates the population of cells into subpopulations is “captured” onto a solid support, e.g., a bead, a membrane. In some embodiments, the solid support is a bead and the bead is incubated with the population of cells obtained from the adipose tissue (e.g., population of cells obtained from the digestion of adipose tissue). In some aspects, the incubation with the bead(s) occurs before or after incubation of these cells with the primary antibody. In some embodiments, the bead(s) are incubated with the population of cells obtained from adipose tissue simultaneously with the primary antibody. Once the cells of the population have been incubated with both the bead(s) and the primary antibody, complexes comprising the bead, the primary antibody, and the target cell or the non-target cell form.

In some embodiments, the beads comprise a protein which binds to the primary antibody. In specific embodiments, the protein is a secondary antibody which specifically binds to the primary antibody, e.g., the Fc region of the primary antibody. In some embodiments, the protein is Protein A, Protein G, Protein A/G, Protein L (e.g., Protein A, Protein G, Protein A/G, Protein L from Staphylococcus aureus).

In some embodiments, the beads are incubated with the population of cells at a bead number to target cell number ratio between about 1:1 and 5:1. In some embodiments, the ratio is about 1:1, about 2:1, about 3:1, about 4:1, or about 5:1.

In certain aspects, the method of obtaining an enriched population of target cells comprises separating the population of cells into a subpopulation comprising target cells and a subpopulation substantially devoid of target cells by removing the complexes comprising the beads and the primary antibody and either the target cell or non-target cell from the cell population which contained the cells, beads, and primary antibody. Methods of removing the beads are known in the art. In some embodiments, the beads are paramagnetic beads and the beads are removed with a magnet. In some embodiments, the beads are separated by centrifugation.

In some embodiments, the complexes comprising the beads and primary antibody further comprises the target cells or the non-target cells. In the embodiments, in which the complexes comprise the non-target cells, the target cells are contained in the solution from which the beads were removed. In some embodiments, no further steps are taken to enrich or purify the target cells.

Release Peptide

In some embodiments, in which the complexes comprise the target cells, the method comprises further steps to release the target cells from the complexes. To this end, in certain aspects, the method comprises incubating the complexes with a release peptide. As used herein, the term “release peptide” is any molecule comprising at least two amino acids connected via a peptide bond which displaces the primary antibody from the target cell.

In some embodiments, the release peptide comprises an epitope which is an epitope of CD34 or an epitope of the primary antibody, e.g., a CDR of the primary antibody. In some aspects, the release peptide is a soluble CD34, (e.g., a soluble fragment of CD34), or a PR34 peptide, which is described in U.S. Pat. Nos. 5,968,753 and 6,017,719. In some embodiments, the release peptide is any of those described in these patents. In some embodiments, the release peptide is one which is provided as part of the Isolex 300i Kit (Baxter, Deerfield, Ill.).

In some embodiments, the concentration of the release peptide is present with the complexes at a final concentration within about 0.01 mg/ml and 10 mg/ml, within about 0.1 mg/ml and about 5 mg/ml, or within about of 1 mg/ml to about 2 mg/ml. In some embodiments, the release peptide is at a final concentration of about 2 mg/ml.

In some embodiments, the release peptide is incubated with the complexes while rotating, shaking, or otherwise moving. In some embodiments, the release peptide is incubated without an movement.

In specific embodiments, the complexes are triturated to increase the efficiency of the release peptide-mediated displacement of the primary antibody from the target cell. In some embodiments, the triturating is accomplished with a syringe, a pipette, or like tool which has a relatively small bore through which cells can pass and which facilitates the breaking of cell clumps formed upon complex formation. In certain aspects, the method comprises triturating for at least about 30 seconds, at least about 1 minute, at least about 5 minutes, at least about 10 minutes, at least about 15 minutes, at least about 25 minutes, at least about 30 minutes, at least about 45 minutes, at least about 60 minutes, at least about 90 minutes, at least about 120 minutes at least about 2 hours, at least about 3 hours, at least about 4 hours. In some aspects, the method comprises triturating for no more than about 10 hours (e.g., no more than about 5 hours).

In certain embodiments, the trituration occurs in the presence of the release peptide. In other embodiments, the trituration occurs without the release peptide present, e.g., the trituration occurs before addition of the release peptide. In some embodiments, the trituration occurs before addition of the release peptide and the trituration and the addition of the release peptide occurs within about 30 seconds, within about 60 seconds, within about 1.5 minutes, within about 2 minutes, within about 5 minutes, within about 10 minutes, within about 15 minutes, within about 30 minutes, within about 45 minutes, within about 60 minutes, of each other.

### Exemplary Embodiments

In specific embodiments, following digestion of the adipose tissue with collagenase or similar enzyme, the digested tissue is then incubated with an anti-CD34 positive antibody or antibody that selects for other epitopes/enzymes/proteins contained on or in the CD34+ cells. The antibody/digest mixture is then subsequently incubated with paramagnetic beads coated with antibody directed against CD34 positive antibody. The bead-antibody complexes with the CD34+ cell-antibody complex, forming a cell-antibody-bead complex. This beaded complex is then separated from the remainder of the adipose tissue digest by use of a magnet. The non-magnetic bound material is then washed away from the bound material and the resultant bound material is then incubated with a peptide, which competes for the anti-CD34 positive antibody. Such peptide has competitive or higher affinity for the anti-CD34 positive antibody and, consequently, the cells are released from the beads, the antibody and the magnetic. This process is enhanced by gentle mechanical agitation (trituration) to break up clumping of the cellular complexes to allow the peptide to remove the antibody and bead, thereby release the cells. The peptide-antibody-bead complexes would then be removed through the use of a magnet.

Antibody selection technology (Isolex 300i, Baxter Healthcare Corp., Deerfield, Ill.) is used to isolate, purify, and harvest human CD34+ stem cells from a patient's blood or bone marrow (U.S. Pat. Nos. 5,536,475; 6,251,295; 5,968,753; 6,017,719). In some aspects, the enrichment process is performed with an Isolex system, for example, the Isolex 300i system or modification thereof (Baxter, Deerfield, Ill.).

Further considerations and additional steps can be taken in some embodiments. Aspects of the invention include combinations of steps described herein. While some embodiments encompass target cells as CD34-positive cells, similar processes may be employed to select for other cells, such as CD271, CD117, CD133 or CD31 positive cells. Such similar processes would involve the use of antibodies such as anti-CD271, anti-CD117, anti-CD133 and antiCD31, for example. The invention contemplates the use of any of these selection processes alone, or in concert with one or more of the other processes such that the final, resultant, enhanced adipose tissue digest would be enriched for one of these cells or mixtures thereof including, but not limited to, an enhanced tissue comprising increased concentrations of all of the cells cited above.

In certain embodiments, the methods described herein obtain a population of target cells from adipose tissue without having to culture the cells for purposes of cell proliferation or expansion. In this regard, in some embodiments, the method is devoid of any steps which expand the population of target cells obtained. In other embodiments, the time in tissue culture is limited and minimal. In some embodiments, the target cells are cultured or plated for no more than 8 hours, no more than 12 hours, no more than 18 hours, no more than 24 hours, no more than 36 hours, or no more than 48 hours.

In some aspects, the target cells are further modified once selected or purified from the population of cells obtained from adipose tissue. In one alternative, the cells are cultured in vitro for purposes of expanding the population of target cells, delivering genes into the target cells, differentiating the target cells, or conjugating a compound, such as a therapeutic agent or a diagnostic agent, to the target cells. Methods of carrying out these further steps are well known in the art. See, for example, Sambrook et al., Molecular Cloning: A Laboratory Manual, Cold Spring Harbor Laboratory Press, Cold Spring Harbor, N.Y., 2001; Ogawa et al., Blood 81: 2844-2853 (1993); U.S. Pat. No. 7,144,731; Li et al, FASEB J 15: 586 (2001); Norol et al., Experimental Hematology 35(4): 653-661 (2007); Verhoeyen and Cosset, Gene Transfer: Delivery and Expression of DNA and RNA, eds. Friedmann and Rossi, Cold Spring Harbor Laboratory Press, Cold Spring Harbor, N.Y., 2007.

Cell Populations

The methods of the invention provide a population of cells from adipose tissue. Accordingly, the invention further provides a population of cells from adipose tissue. In some embodiments, the population of cells of the invention is (i) a population of primary cells obtained or derived from adipose tissue and which have had little to no time in tissue culture for the purposes of cell expansion, (ii) comprises adherent and non-adherent cells, (iii) comprises CD34-positive cells, (iv) or a combination of (i) through (iv). In some embodiments in which the population of cells of the invention comprises CD34-positive cells, at least 25% (e.g., at least 30%, at least 40%, at least 50%, at least 60%, at least 70%, at least 80%, at least 90%, at least 95%) of the cells of the population are CD34-positive cells. In some embodiments in which the population of cells is a population of primary cells obtained or derived from adipose tissue and which have had little to no time in tissue culture for the purposes of cell expansion, the cells have been in tissue culture is limited and minimal. In some embodiments, the cells have been cultured or plated for no more than 8 hours, no more than 12 hours, no more than 18 hours, no more than 24 hours, no more than 36 hours, or no more than 48 hours. In some embodiments, the cells have never been cultured or plated.

In some embodiments, the population of cells is the population obtained upon digesting adipose tissue, with or without selection steps, in accordance with any of the methods described herein.

In some embodiments, the cell populations of the invention are substantially isolated. The term “isolated” as used herein means having been removed from its natural environment. Because the cells of the population have been obtained and removed from adipose tissue, the cells in most embodiments are considered as “isolated” cells.

In some embodiments, the cell populations of the invention are substantially purified or enriched or selected. The terms “purified,” “enriched,” and “selected” as used herein means having been increased in purity as a result of being separated from other components of the original composition. It is recognized that “purity” or “enrichment” or “selection” is a relative term, and not to be necessarily construed as absolute purity or absolute enrichment or absolute selection. In some aspects, the purity is at least about 50%, is greater than 60%, 70%, 80%, or 90%, is about 91%, about 92%, about 93%, about 94%, about 95%, about 96%, about 97%, about 98%, about 99% or is approximately 100%. In some embodiments, the enrichment or selection is a 1.5-fold, 2-fold, 3-fold, 4-fold, 5-fold, 6-fold, 7-fold, 8-fold, 9-fold, 10-fold, 50-fold, 100-fold, 1000-fold enrichment or selection, as compared to the original composition. In some embodiments, the percentage of target cells in the enriched population is about 1.5 to about 5-fold more than the percentage of target cells in the population of cells before selection or purification. In some embodiments, the percentage of target cells in the enriched population is about 1.5 to about 5-fold more than the percentage of target cells in the population of cells before antibody-based selection but after digestion with the enzyme solution.

In some embodiments, the population of cells of the invention are substantially purified of adipocytes, white blood cells, and/or red blood cells, i.e., the population of cells obtained from adipose tissue are substantially devoid of adipocytes, white blood cells, and/or red blood cells. In some aspects, the population of cells of the invention are substantially purified of debris or dead cells.

In some embodiments, the population of cells is one which has undergone positive selection steps. In some embodiments, the population of cells is one which has undergone negative selection steps. In some embodiments, the population of cells is one which has undergone both positive and negative selection steps. In some aspects, the population of cells of the invention is an enriched population of target cells. In some embodiments, the population of cells of the invention is an enriched population of target cells obtained from adipose tissue in accordance with any of the methods described herein.

In some embodiments, the population of cells (e.g., the enriched population of target cells) is a substantially homogenous population of cells. In some aspects, the population of cells (e.g., the enriched population of target cells) is a clonal population of target cells, wherein each cell of the population is genetically indistinct from another cell of the population.

In some embodiments the population of cells (e.g., the enriched population of target cells) is a heterogeneous population of cells. In some embodiments, the heterogeneous population of cells comprises only target cells, but the population is not a clonal population, e.g., not genetically indistinct from each other. In some embodiments, a substantial portion of the population of cells expresses one or more common cells markers, e.g., CD34, but the expression levels of other cell markers is different among the cells of the population. In some aspects, the target cells are CD34+ cells and the CD34+ cells are adipocytes, lymphocytes, macrophages, mesenchymal stem cells.

In some embodiments, the heterogeneous population comprises other types of cells, cells other than the target cells. In some aspects the heterogeneous population of cells comprises, in addition to the target cells, a white blood cells (a white blood cells of myeloid lineage or lymphoid lineage), a red blood cell, an endothelial cell, circulating endothelial precursor cells, an epithelial cell, a kidney cell, a lung cell, an osteocyte, a myelocyte, a neuron, smooth muscle cells.

In some specific aspects, the population comprises a variety of cell types and a substantial portion of the population comprises a common phenotype, biological function, or state of maturity or differentiation. In specific aspects, at least at least 25% (e.g., at least 30%, at least 40%, at least 50%, at least 60%, at least 70%, at least 80%, at least 90%, at least 95%) of the cells in the population are stem cells (e.g., adipose stromal stem cells, mesenchymal stem cells, hematopoietic stem cells). In specific aspects, at least at least 25% (e.g., at least 30%, at least 40%, at least 50%, at least 60%, at least 70%, at least 80%, at least 90%, at least 95%) of the cells in the population are endothelial cells.

In some embodiments, the population of cells comprises adherent cells or nonadherent cells. In some embodiments, the population of cells obtained from adipose tissue comprises both adherent and non adherent cells.

Suitable methods of isolating, purifying, selecting, enriching cells having a particular phenotype are known in the art and include, for instance, methods using optical flow sorters (e.g., fluorescence-activated cell sorting (FACS)) and methods using non-optical flow sorters (e.g., magnetic-activated cell sorting) and the methods described herein.

Cell Markers

The population of cells obtained from adipose tissue (including the enriched population of target cells) is in some embodiments a heterogeneous cell population, wherein at least 25% (e.g., at least 30%, at least 40%, at least 50%, at least 60%, at least 70%, at least 80%, at least 90%, at least 95%) of the cells in the population have a particular phenotype (e.g., is positive for expression of a cell marker and/or negative for expression of a cell marker). In some embodiments of the invention, at least 25% (e.g., at least 30%, at least 40%, at least 50%, at least 60%, at least 70%, at least 80%, at least 90%, at least 95%) of the cells of the population obtained from adipose tissue are CD34-positive cells. In some embodiments of the invention, at least 25% (e.g., at least 30%, at least 40%, at least 50%, at least 60%, at least 70%, at least 80%, at least 90%, at least 95%) of the cells of the population are CD45-negative cells. In some embodiments of the invention, at least 25% (e.g., at least 30%, at least 40%, at least 50%, at least 60%, at least 70%, at least 80%, at least 90%, at least 95%) of the cells are positive for expression of any of the following cell markers: CD140b, CD90, CD31, CD105, CD73, CD144, CD105, CD106, CD44, CD146. In some embodiments of the invention, at least 25% (e.g., at least 30%, at least 40%, at least 50%, at least 60%, at least 70%, at least 80%, at least 90%, at least 95%) of the cells are negative for expression of any of the following cell markers: CD140b, CD90, CD31, CD105, CD73, CD144, CD105, CD106, CD44, CD146.

Methods of Preparing Pharmaceutical Compositions

The populations of cells, including the enriched population of target cells) obtained from adipose tissue are believed to have therapeutic value. In this regard, the invention further provides a method of preparing a pharmaceutical composition comprising cells for administration to a patient, comprising formulating the population of cells (e.g., the enriched population of target cells) obtained in accordance with any of the methods described herein with a pharmaceutically acceptable carrier.

In some aspects, the donor of the adipose tissue is the same as the patient. In this regard, the cells (e.g., target cells) are considered “autologous” to the patient. In some embodiments, the donor of the cells (e.g., target cells) is different from the patient, but the donor and patient are of the same species. In this regard, the cells (e.g., target cells) are considered as “allogeneic.”

In certain embodiments, the cells (e.g., target cells) have been freshly obtained from the adipose tissue. In specific aspects, the cells have been cultured or plated only to a limited extent, e.g., not more than 4 hours, 6 hours, 8 hours, 12 hours, 18 hours, 24 hours, 36 hours, or 48 hours. In other aspects, the cells have never been cultured or plated before formulated with a pharmaceutically acceptable carrier.

Pharmaceutical Compositions

The invention accordingly provides pharmaceutical compositions comprising the population of cells (e.g., the enriched population of target cells) obtained through the any of the methods described herein formulated with a pharmaceutically acceptable carrier. In this regard, the invention provides a pharmaceutical composition comprising the cells (e.g., target cells) and a pharmaceutically acceptable carrier. The carrier is any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. In one aspect the pharmaceutically acceptable carrier is one which is chemically inert to the cells, e.g., the target cells, and one which has no detrimental side effects or toxicity under the conditions of use. The choice of carrier will be determined in part by the particular type of cells of the pharmaceutical composition, as well as by the particular route used to administer the pharmaceutical composition. Accordingly, there are a variety of suitable formulations of the pharmaceutical composition of the invention.

Routes of Administration

In some embodiments, the pharmaceutical composition comprising the cells (e.g., target cells) is formulated for parenteral administration, subcutaneous administration, intravenous administration, intramuscular administration, intraarterial administration, intrathecal administration, or interperitoneal administration. In other embodiments, the pharmaceutical composition is administered via nasal, spray, oral, aerosol, rectal, or vaginal administration.

Methods of administering cells (e.g., target cells) are known in the art. See, for example, any of U.S. Pat. Nos. 5,423,778, 5,550,050, 5,662,895, 5,800,828, 5,800,829, 5,811,407, 5,833,979, 5,834,001, 5,834,029, 5,853,717, 5,855,619, 5,906,827, 6,008,035, 6,012,450, 6,049,026, 6,083,523, 6,206,914, 6,303,136, 6,306,424, 6,322,804, 6,352,555, 6,368,612, 6,479,283, 6,514,522, 6,534,052, 6,541,024, 6,551,338, 6,551,618, 6,569,147, 6,579,313, 6,599,274, 6,607,501, 6,630,457, 6,648,849, 6,659,950, 6,692,738, 6,699,471, 6,736,799, 6,752,834, 6,758,828, 6,787,357, 6,790,455, 6,805,860, 6,852,534, 6,863,900, 6,875,441, 6,881,226, 6,884,427, 6,884,428, 6,886,568, 6,918,869, 6,933,281, 6,933,286, 6,949,590, 6,960,351, 7,011,828, 7,031,775, 7,033,345, 7,033,603, 7,049,348, 7,070,582, 7,074,239, 7,097,832, 7,097,833, 7,135,172, 7,145,055, 7,157,080, 7,166,280, 7,176,256, 7,244,242, 7,452,532, 7,470,425, and 7,494,644.

Parenteral

In some embodiments, the pharmaceutical composition described herein is formulated for parenteral administration. For purposes of the invention, parenteral administration includes, but is not limited to, intravenous, intraarterial, intramuscular, intracerebral, intracerebroventricular, intracardiac, subcutaneous, intraosseous, intradermal, intrathecal, intraperitoneal, intravesical, and intracavernosal injections or infusions.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The pharmaceutical composition are in various aspects administered via a physiologically acceptable diluent in a pharmaceutical carrier, such as a sterile liquid or mixture of liquids, including water, saline, aqueous dextrose and related sugar solutions, a glycol, such as propylene glycol or polyethylene glycol, glycerol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which are optionally used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

The parenteral formulations in some embodiments contain preservatives or buffers. In order to minimize or eliminate irritation at the site of injection, such compositions optionally contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations are in various aspects presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions are in certain aspects prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Cell Delivery Matrices

In some embodiments, the cells (e.g., target cells) are administered via a cell delivery matrix. The cell delivery matrix in certain embodiments comprises any one or more of polymers and hydrogels comprising collagen, fibrin, chitosan, MATRIGEL, polyethylene glycol, dextrans including chemically crosslinkable or photocrosslinkable dextrans, and the like. In certain embodiments, the cell delivery matrix comprises one or more of: collagen, including contracted and non-contracted collagen gels, hydrogels comprising, for example, but not limited to, fibrin, alginate, agarose, gelatin, hyaluronate, polyethylene glycol (PEG), dextrans, including dextrans that are suitable for chemical crosslinking, photocrosslinking, or both, albumin, polyacrylamide, polyglycolyic acid, polyvinyl chloride, polyvinyl alcohol, poly(n-vinyl-2-pyrollidone), poly(2-hydroxy ethyl methacrylate), hydrophilic polyurethanes, acrylic derivatives, pluronics, such as polypropylene oxide and polyethylene oxide copolymer, 35/65 Poly(epsilon-caprolactone)(PCL)/Poly(glycolic acid) (PGA), Panacryl® bioabsorbable constructs, Vicryl® polyglactin 910, and self-assembling peptides and non-resorbable materials such as fluoropolymers (e.g., Teflon® fluoropolymers), plastic, and metal.

The matrix in some instances comprises non-degradable materials, for example, but not limited to, expanded polytetrafluoroethylene (ePTFE), polytetrafluoroethylene (PTFE), polyethyleneterephthalate (PET), poly(butylenes terephthalate (PBT), polyurethane, polyethylene, polycabonate, polystyrene, silicone, and the like, or selectively degradable materials, such as poly (lactic-co-glycolic acid; PLGA), PLA, or PGA. (See also, Middleton et al., Biomaterials 21:2335 2346, 2000; Middleton et al., Medical Plastics and Biomaterials, March/April 1998, at pages 30 37; Handbook of Biodegradable Polymers, Domb, Kost, and Domb, eds., 1997, Harwood Academic Publishers, Australia; Rogalla, Minim. Invasive Surg. Nurs. 11:6769, 1997; Klein, Facial Plast. Surg. Clin. North Amer. 9:205 18, 2001; Klein et al., J. Dermatol. Surg. Oncol. 1 1:337 39, 1985; Frey et al., J. Urol. 154:812 15, 1995; Peters et al., J. Biomed. Mater. Res. 43:422 27, 1998; and Kuijpers et al., J. Biomed. Mater. Res. 51:13645, 2000).

The matrix in some embodiments includes biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. In certain aspects, the matrix is pretreated with the cells (e.g., target cells). In other embodiments, the matrix is populated with cells (e.g., target cells) in close association to the matrix or its spaces. The cells (e.g., target cells) can adhere to the matrix or can be entrapped or contained within the matrix spaces. In certain aspects, the matrix-cells (e.g., target cells) complexes in which the cells are growing in close association with the matrix and when used therapeutically, growth, repair, and/or regeneration of the patient's own kidney cells is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like. In some embodiments, the matrices form in vivo, or even more preferably in situ, for example in situ polymerizable gels can be used in accordance with the invention. Examples of such gels are known in the art or the like.

The cells (e.g., target cells) in some embodiments are seeded on a three-dimensional framework or matrix, such as a scaffold, a foam or hydrogel and administered accordingly. The framework in certain aspects are configured into various shapes such as substantially flat, substantially cylindrical or tubular, or can be completely free-form as may be required or desired for the corrective structure under consideration. Two or more substantially flat frameworks in some aspects are laid atop another and secured together as necessary to generate a multilayer framework.

Examples of matrices, for example scaffolds which may be used for aspects of the invention include mats (woven, knitted, and more preferably nonwoven) porous or semiporous foams, self assembling peptides and the like. Nonwoven mats may, for example, be formed using fibers comprised of natural or synthetic polymers. In some embodiments, absorbable copolymers of glycolic and lactic acids (PGA/PLA), sold under the tradename VICRYL® (Ethicon, Inc., Somerville, N.J.) are used to form a mat. Foams, composed of, for example, poly(epsilon-caprolactone)/poly(glycolic acid) (PCL/PGA) copolymer, formed by processes such as freeze-drying, or lyophilization, as discussed in U.S. Pat. No. 6,355,699, can also serve as scaffolds. Gels also form suitable matrices, as used herein. Examples include in situ polymerizable gels, and hydrogels, for example composed of self-assembling peptides. These materials are used in some aspects as supports for growth of tissue. In situ-forming degradable networks are also suitable for use in the invention (see, e.g., Anseth, K. S. et al., 2002, J. Controlled Release 78: 199-209; Wang, D. et al., 2003, Biomaterials 24: 3969-3980; U.S. Patent Publication 2002/0022676 to He et al.). These materials are formulated in some aspects as fluids suitable for injection, then may be induced by a variety of means (e.g., change in temperature, pH, exposure to light) to form degradable hydrogel networks in situ or in vivo.

In some embodiments, the framework is a felt, which is comprised of a multifilament yarn made from a bioabsorbable material, e.g., PGA, PLA, PCL copolymers or blends, or hyaluronic acid. The yarn in certain aspects is made into a felt using standard textile processing techniques consisting of crimping, cutting, carding and needling. The cells (e.g., target cells) in certain aspects are seeded onto foam scaffolds that may be composite structures. In addition, the three-dimensional framework are molded in some aspects into a useful shape, such as a specific structure in or around the kidney to be repaired, replaced, or augmented.

The framework in certain aspects is treated prior to inoculation of the cells (e.g., target cells) in order to enhance cell attachment. For example, prior to inoculation with the cells (e.g., target cells), nylon matrices are treated with 0.1 molar acetic acid and incubated in polylysine, PBS, and/or collagen to coat the nylon. Polystyrene is some aspects is similarly treated using sulfuric acid.

In additional embodiments, the external surfaces of the three-dimensional framework is modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

The scaffold in some embodiments comprises materials that render it non-thrombogenic. These materials in certain embodiments promote and sustain endothelial growth, migration, and extracellular matrix deposition. Examples of such materials include but are not limited to natural materials such as basement membrane proteins such as laminin and Type IV collagen, synthetic materials such as ePTFE, and segmented polyurethaneurea silicones, such as PURSPAN® (The Polymer Technology Group, Inc., Berkeley, Calif.). These materials can be further treated to render the scaffold non-thrombogenic. Such treatments include anti-thrombotic agents such as heparin, and treatments which alter the surface charge of the material such as plasma coating.

The pharmaceutical composition comprising the cells (e.g., target cells) in certain embodiments comprises any of the components of a cell delivery matrix, including any of the components described herein.

In some embodiments, the pharmaceutical composition comprises stem cells. Administration of stem cells to animals with ischemic injury is described in U.S. Pat. No. 5,980,887.

In aspects of the invention, the target cells are adipose derived CD34+ cells. The enhanced CD34+ cells mixture devoid of all or substantially all of the processing reagents may then be placed in a media suitable for therapeutic injection to a patient. Such media are generally known to those skilled in the art, and may include, but are not limited to, irrigating solutions, cell culture solutions and the like. In some aspects, the CD34+ cells are delivered to a patient by one of several means. In some embodiments, the CD34+ cells are delivered intramuscularly, intra-peritoneally, intra-cranially, intra-vascularly, intravenously, between tissue components such as fractured or broken bone or cartilage.

Possible delivery options of target include but are not limited to: direct injection (needle and syringe); injection catheter (deeper tissue); spray for surface; implanting pre-made fibrin (subcutaneous or deeper within tissue beds) in conjunction with bioscaffolds. (both internal and external). In aspects of the invention, the target body site for delivery can be heart, limb, eye, brain, kidney, nerve, liver, kidney, heart, lung, eye, organs of the gastrointestinal tract, skin, and brain.

Dose

For purposes herein, the amount or dose of the pharmaceutical composition administered is sufficient to effect, e.g., a therapeutic or prophylactic response, in the subject or animal over a reasonable time frame. For example, the dose of the pharmaceutical composition is sufficient to treat or prevent a disease or medical condition in a period of from about 12 hours, about 18 hours, about 1 to 4 days or longer, e.g., 5 days, 6 days, 1 week, 10 days, 2 weeks, 16 to 20 days, or more, from the time of administration. In certain embodiments, the time period is even longer. The dose is determined by the efficacy of the particular pharmaceutical composition and the condition of the animal (e.g., human), as well as the body weight of the animal (e.g., human) to be treated.

Many assays for determining an administered dose are known in the art. In some embodiments, an assay which comprises comparing the extent to which cells (e.g., target cells) are localized to the injured site upon administration of a given dose of such cells (e.g., target cells) to a mammal among a set of mammals each of which is given a different dose of the cells (e.g., target cells) is used to determine a starting dose to be administered to a mammal. The extent to which cells (e.g., target cells) are localized to the injured site upon administration of a certain dose can be assayed by methods known in the art.

Also, an assay which comprises comparing the extent to which cells (e.g., target cells) cause reperfusion of an injured hindlimb upon administration of a given dose of such cells (e.g., target cells) to a mammal among a set of mammals each of which is given a different dose of the cells (e.g., target cells) is used to determine a starting dose to be administered to a mammal. The extent to which cells (e.g., target cells) cause reperfusion of an injured hindlimb upon administration of a certain dose can be assayed by methods known in the art and are described herein.

The dose of the pharmaceutical composition also will be determined by the existence, nature and extent of any adverse side effects that might accompany the administration of a particular pharmaceutical composition. Typically, the attending physician will decide the dosage of the pharmaceutical composition with which to treat each individual patient, taking into consideration a variety of factors, such as age, body weight, general health, diet, sex, therapeutic agent(s) (e.g., cells (e.g., target cells) of the pharmaceutical composition to be administered, route of administration, and the severity of the condition being treated. By way of example and not intending to limit the invention, the dose of the pharmaceutical composition can be such that at least about 0.5×106 (e.g., at least about 1×106, at least about 1.5×106, at least about 2×106, at least about 2.5×106, at least about 3.0×106, at least about 5.0×106, at least about 107, at least about 108) cells (e.g., target cells) are administered to the patient.

Timing of Administration

In certain embodiments of the invention, administration of the cells (e.g., target cells) is delayed; that is, the cells (e.g., target cells) are not administered immediately after injury (e.g., not before about 30 minutes, not before about 1 hour, not before about 2 hours, not before about 3 hours, not before about 4 hours, not before about 5 hours, not before about 6 hours, not before about 7 hours, not before about 8 hours, not before about 9 hours, not before about 10 hours, not before about 11 hours, or not before about 12 hours post-injury).

In some aspects of the invention, the cells (e.g., target cells) are administered to the patient at the beginning of the repair phase of the injury. In some embodiments, the cells (e.g., target cells) are administered at least about 12 hours (e.g., at least about 14 hours, at least about 16 hours, at least about 18 hours, at least about 20 hours, at least about 21 hours, at least about 22 hours, at least about 23 hours, at least about 24 hours, at least about 25 hours, at least about 26 hours, at least about 28 hours, at least about 30 hours, at least about 32 hours, at least about 32 hours, at least about 34 hours, at least about 36 hours, at least about 38 hours, at least about 40 hours, at least about 42 hours, at least about 44 hours, at least about 46 hours, at least about 48 hours, at least about 50 hours, at least about 52 hours, at least about 54 hours, at least about 56 hours, at least about 58 hours, at least about 60 hours, at least about 62 hours, at least about 64 hours, at least about 66 hours, at least about 68 hours, at least about 70 hours, at least about 72 hours) post-injury.

In further embodiments, the cells (e.g., target cells) are administered to the patient at a timepoint as described above and before about 14 days (e.g., before about 13 days, before about 12 days, before about 11 days, before about 10 days, before about 9 days, before about 8 days, before about 7 days, before about 6 days, before about 5 days, before about 4 days, before about 3 days) post injury. In some embodiments, the cells (e.g., target cells) are administered to the patient at about 24 hours post-injury, or some time thereafter, but before about 14 days post-injury.

In some aspects, the cells (e.g., target cells) are administered after X post-injury and before Y post-injury, wherein X is selected from a group consisting of about 20 h, about 21 h, about 22 h, about 23 h, about 24 h, about 25 h, about 26 h, about 27 h, about 28 h, about 29 h, about 30 h, about 31 h, about 32 h, about 33 h, about 34 h, about 35 h, about 36 h, about 40 h, about 48 h, about 52 h, about 58 h, about 64 h, about 72 h, about 3.5 d, about 4 d, about 5 d, about 6 d, about 1 week, about 8 d, about 9 d, about 10 d, wherein Y is selected from a group consisting of about 16 d, about 15 d, about 14 d, about 13 d, about 12 d, about 11 d, about 10 d, about 9 d, about 8 d, about 1 week, and wherein X is less than Y. In some aspects of the invention, the cells (e.g., target cells) are administered about 20, about 21, about 22, about 23, about 24 hours post-injury.

In some embodiments of the invention, the cells (e.g., target cells) are administered to the patient more than once. The cells (e.g., target cells) may be administered once daily, twice daily, 3×, 4× daily, once weekly, once every 2, 3, 4, 5, 6, 8, 9, 10, 11, 12, 13, or 14 days, or once monthly. In some embodiments, the cells (e.g., target cells) are administered after about 24 hours (e.g., at 24 hours) post-injury and administered again after about 48 hours (e.g., at 48 hours) post-injury.

Controlled Release Formulations

The pharmaceutical composition are in certain aspects modified into a depot form, such that the manner in which the pharmaceutical composition is released into the body to which it is administered is controlled with respect to time and location within the body (see, for example, U.S. Pat. No. 4,450,150). Depot forms are in various aspects, an implantable composition comprising cells (e.g., target cells) and a porous or non-porous material, such as a polymer, wherein the cells (e.g., target cells) is encapsulated by or diffused throughout the material and/or degradation of the non-porous material. The depot is then implanted into the desired location within the body and the cells (e.g., target cells) are released from the implant at a predetermined rate.

Accordingly, the pharmaceutical composition in certain aspects is modified to have any type of in vivo release profile. In some aspects of the invention, the pharmaceutical composition is an immediate release, controlled release, sustained release, extended release, delayed release, or bi-phasic release formulation.

Conjugates

In some embodiments of the invention, the cells (e.g., target cells) are attached or linked to a second moiety, such as, for example, a therapeutic agent or a diagnostic agent. The cells (e.g., target cells) of these embodiments act as a targeting agent, since the cells (e.g., target cells) are able to specifically localize to injured kidney tissue. Accordingly, the invention provides in one aspect a composition comprising cells (e.g., target cells) attached to a therapeutic agent of a diagnostic agent. Suitable therapeutic agents and diagnostic agents for purposes herein are known in the art and include, but are not limited to, any of those mentioned herein.

Combinations

The pharmaceutical compositions described herein, including the conjugates, are administered by itself in some embodiments. In other embodiments, the pharmaceutical compositions, including the conjugates, are administered in combination with other therapeutic or diagnostic agents. In some embodiments, the pharmaceutical composition is administered with another therapeutic agent known to treat a renal disease or renal medical condition, including, for example, a cytokine or growth factor, an anti-inflammatory agent, a TLR2 inhibitor, a ATF3 gene or gene product, and a mineralocorticoid receptor blocker (e.g., spironolactone), a lysophosphatidic acid, 2-methylaminochroman (e.g., U83836E), a 21-aminosteroid (e.g., lazoroid (U74389F)), trimetazidine, angiotensin converting enzyme (ACE) inhibitors or angiotensin receptor blockers (ARB), and suramin.

In some embodiments, the cells (e.g., target cells) are administered with other additional therapeutic agents, including, but not limited to, antithrombogenic agents, anti-apoptotic agents, anti-inflammatory agents, immunosuppressants (e.g., cyclosporine, rapamycin), antioxidants, or other agents ordinarily used in the art to treat kidney damage or disease such as eprodisate and triptolide, an HMG-CoA reductase inhibitor (e.g., simvastatin, pravastatin, lovastatin, fluvastatin, cerivastatin, and atorvastatin), cell lysates, soluble cell fractions, membrane-enriched cell fractions, cell culture media (e.g., conditioned media), or extracellular matrix trophic factors (e.g., hepatocyte growth factor (HGF), bone morphogenic protein-7 (BMP-7), transforming growth factor beta (TGF-β), matrix metalloproteinase-2 (MMP-2), and basic fibroblast growth factor (bFGF).

In certain embodiments, the subpopulation of target cells are combined with other stem cells selected from the group consisting of totipotent stem cells, pluripotent stem cells, hematopoietic stem cells, and any other stem cells. In some embodiments, the target cells are combined with non-hematopoietic stem cells, such as, but not limited to mesenchymal cells. The target cells in some embodiments are combined with scaffolds such as but not limited to fibrin, collagen, or polyethylene glycol (PEG).

The selected cells in some embodiments is used in concert with various growth factors or other bioactive agents. They could be modified using gene therapy for use as up or down regulators.

Uses

The pharmaceutical compositions described herein are believed to be useful in the therapeutic treatment of many diseases and medical conditions. Accordingly, the method additionally provides a method of treating a disease or medical condition comprising administering to the patient any of the pharmaceutical compositions described herein in an amount effective to treat the disease or medical condition. The term “treat,” as well as words stemming therefrom, as used herein, does not necessarily imply 100% or complete amelioration of a targeted condition. Rather, there are varying degrees of a therapeutic effect which one of ordinary skill in the art recognizes as having a benefit. In this respect, the methods described herein provide any amount or any level of therapeutic benefit of a kidney injury and therefore “treat” the injury.

In some aspects, the disease or medical condition is chronic myocardial ischemia, critical limb ischemia, acute myocardial infarction, cardiovascular disease, diabetes, autoimmune diseases, stroke, brain and/or spinal cord injury, burn injury, bone defects, renal ischemia, and macular degeneration. In some embodiments, the pharmaceutical compositions are used to treat tissue damage due to ischemia, blood flow loss, lacerations, extremes of temperature, trauma, or metabolic or genetic disease.

In some aspects, in which the pharmaceutical composition comprises adipose derived stem cells, the method comprises providing therapeutic effects such as but not limited to: proangiogenic effects to combat ischemia, producing cell, tissue, and/or organ regeneration, wound healing, differentiation, reconstitution of blood supply, decrease of apoptosis, paracrine signaling, and immunomodulation. In some aspects, in which cells are selected using the positive selection procedure using antibodies directed to CD34 and CD271, the method treats inflammation. In aspects in which the pharmaceutical composition comprises CD34+ cells, the method provides anti apoptotic effects.

Cells within adipose tissue have been shown to have differentiation potential and could therefore be used for the repair and regeneration of multiple tissues and for multiple injury types. These cells may also be helpful in the area of cosmetic surgery. Due to the ease of processing and the relatively non invasive nature of liposuction, these cells could be selected for their abilities to repair tissue, or used in conjunction with wound healing agents to speed recovery.

Kidney Injuries

In various aspect of the invention, methods provided are intended to treat kidney injury in the patient which is any injury to the kidney caused by any one or more of: ischemia, exposure to a toxin, use of an angiotensin-converting enzyme inhibitor (ACEI) or angiotensin II receptor blocker, a blood transfusion reaction, an injury or trauma to muscle, surgery, shock, hypotension, or any of the causes of ARF or chronic kidney disease, as further described herein.

The targeted kidney injury comprises injury to any tissue found within the kidney, including, but not limited to, a tissue of the medulla, cortex, renal pyramid, interlobar artery, renal artery, renal vein, renal hilum, renal pelvis, ureter, minor calyx, renal capsule, inferior renal capsule, superior renal capsule, interlobar vein, nephron, major calyx, renal papilla, glomerulus, Bowman's capsule, and renal column, which tissue is sufficiently damaged to result in a partial or complete loss of function. The injured kidney tissue comprises any one or more of distinct cell types which occur in the kidney, including, but not limited to, kidney glomerulus parietal cells, kidney glomerulus podocytes, intraglomerular mesangial cells, endothelial cells of the glomerulus, kidney proximal tubule brush border cells, loop of Henle thin segment cells, thick ascending limb cells, kidney distal tubule cells, kidney collecting duct cells, and interstitial kidney cells. In certain embodiments of the invention, the kidney injury comprises injury to a kidney peritubular microvasculature. In certain aspects, the kidney injury comprises injury to a peritubular capillary. In some embodiments, the kidney injury comprises injury to tubule (tubular) epithelial cells.

Prevention of Renal Disease and Renal Medical Conditions

While the kidney has tremendous capacity for self-repair or self-regeneration, a kidney injury often leads to an increased predisposition to a renal disease or renal medical condition. It is theorized that the method of treating a kidney injury in a patient provided herein allows for successful repair and regeneration of the kidney, so that the patient does not have an increased predisposition to a renal disease or renal medical condition. Therefore, the invention further provides a method of preventing a renal disease or renal medical condition in a patient comprising a kidney injury. The method comprises administering to the patient cells (e.g., target cells) in an amount effective to prevent the renal disease or renal medical condition. In some embodiments, the amount is effective to treat the kidney injury, e.g., an amount effective to restore kidney function, to regenerate kidney peritubular microvasculature.

As used herein, the term “prevent” as well as words stemming therefrom, does not necessarily imply 100% or complete prevention. Rather, there are varying degrees of prevention of which one of ordinary skill in the art recognizes as having a potential benefit. In this respect, the methods of preventing described herein provide any amount or any level of prevention of renal disease or renal medical condition. In various aspects, the method of preventing is a method of delaying, slowing, reducing, or attenuating the onset, development, occurrence, or progression of the renal disease or renal medical condition, or a symptom or condition thereof.

In some embodiments, the renal disease or renal medical condition prevented is acute renal failure, chronic kidney disease, renal interstitial fibrosis, diabetic nephropathy, glomerulonephritis, hydronephrosis, interstitial nephritis, kidney stones (nephrolithiasis), kidney tumors (e.g., Wilms tumor, renal cell carcinoma), lupus nephritis, minimal change disease, nephrotic syndrome, pyelonephritis, renal failure (e.g., other than acute renal failure and chronic kidney disease).

Acute Renal Failure

The term “acute renal failure” as used herein is synonymous with “acute kidney injury” or “ARF” and refers to a rapid loss of renal function due to damage to the kidneys. ARF is a complex syndrome marked by abrupt changes in the levels of nitrogenous (e.g., serum creatine and/or urine output) and non-nitrogenous waste products that are normally excreted by the kidney. The symptoms and diagnosis of ARF are known in the art. See, for example, Acute Kidney Injury, Contributions to Nephrology, Vol. 156, vol. eds. Ronco et al., Karger Publishers, Basel, Switzerland, 2007, and Bellomo et al., Crit Care 8(4): R204-R212, 2004.

In various aspects, the ARF is a pre-renal ARF, an intrinsic ARF, or a post-renal ARF, depending on the cause. In this regard, the pre-renal ARF may be caused by one or more of: hypovolemia (e.g., due to shock, dehydration, fluid loss, or excessive diruretic use), hepatorenal syndrome, vascular problems (e.g., atheroembolic disease, renal vein thrombosis, relating to nephrotic syndrome), infection (e.g., sepsis), severe burns, sequestration (e.g., due to pericarditis, pancreatitis), and hypotension (e.g., due to antihypertensiveness, vasodilator use).

The intrinsic ARF may be caused by one or more of: toxins or medications (e.g., NSAIDs, aminoglycoside antibiotics, iodinated contrast, lithium, phosphate nephropathy (e.g., associated with colonoscopy bowel preparation with sodium phosphates), rhabdomyolysis (e.g., caused by injury (e.g., crush injury or extensive blunt trauma), statins, stimulant use), hemolysis, multiple myeloma, acute glomerulonephritis.

The post-renal ARF may be caused by one or more of: medication (e.g., anticholinergics), benign prostatic hypertrophy or prostate cancer, kidney stones, abdominal malignancy (e.g., ovarian cancer, colorectal cancer), obstructed urinary catheter, and drugs that cause crystalluria or myoglobulinuria, or cystitis.

ARF may be caused by ischemia, a toxin, use of an angiotensin-converting enzyme inhibitor (ACEI) or angiotensin II receptor blocker, a blood transfusion reaction, an injury or trauma to muscle, surgery, shock, and hypotension in the patient. The toxin which causes ARF can be an antifungal or a radiographic dye. Also, in some embodiments, ARF involves acute tubular necrosis or renal ischemia reperfusion injury.

Chronic Kidney Disease

In some embodiments of the methods of preventing a renal disease or renal medical condition, the renal disease is chronic kidney disease (CKD). As used herein, “chronic kidney disease,” which is also known as “chronic renal disease,” refers to a progressive loss of renal function over a period of months or years. The CKD being treated is any stage, including, for example, Stage 1, Stage 2, Stage 3, Stage 4, or Stage 5 (also known as established CKD, end-stage renal disease (ESRD), chronic kidney failure (CKF), or chronic renal failure (CRF)).

The CKD may be caused by any one of a number of factors, including, but not limited to, acute kidney injury, causes of acute kidney injury, Type 1 and Type 2 diabetes mellitus leading to diabetic nephropathy, high blood pressure (hypertension), glomerulonephritis (inflammation and damage of the filtration system of the kidneys), polycystic kidney disease, use (e.g., regular and over long durations of time) of analgesics (e.g., acetaminophen, ibuprofen) leading to analgesic nephropathy, atherosclerosis leading to ischemic nephropathy, obstruction of the flow of urine by stones, an enlarged prostate, strictures (narrowings), HIV infection, sickle cell disease, heroin abuse, amyloidosis, kidney stones, chronic kidney infections, and certain cancers.

Prevention of Non-Renal Diseases and Non-Renal Medical Conditions

Chronic kidney disease has been identified as a leading independent risk factor for cardiovascular diseases and cardiovascular mortality. It is theorized that the administration of cells (e.g., target cells) as described herein is furthermore useful for preventing diseases or medical conditions other than renal diseases and renal medical conditions. Accordingly, a method of preventing a non-renal disease or non-renal medical condition which is caused by or associated with a renal disease or renal medical condition in a patient comprising a kidney injury is further provided herein. The method comprises administering to the patient cells (e.g., target cells) in an amount effective to prevent the non-renal disease or non-renal medical condition. In certain embodiments, the non-renal disease or non-renal medical condition is cardiovascular disease.

In some embodiments, the disease or medical condition treated by the method provided herein is an autoimmune disease. For purposes herein, “autoimmune disease” refers to a disease in which the body produces an immunogenic (i.e., immune system) response to some constituent of its own tissue. In other words the immune system loses its ability to recognize some tissue or system within the body as “self and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

In some embodiments, the disease is a cancer. In specific embodiments, the cancer is selected from the group consisting of: acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

Patient Types

With regard to the inventive methods described herein, the patient is any host. In some embodiments, the host is a mammal. As used herein, the term “mammal” refers to any vertebrate animal of the mammalia class, including, but not limited to, any of the monotreme, marsupial, and placental taxas. In some embodiments, the mammal is one of the mammals of the order Rodentia, such as mice and hamsters, and mammals of the order Logomorpha, such as rabbits. In certain embodiments, the mammals are from the order Carnivora, including Felines (cats) and Canines (dogs). In certain embodiments, the mammals are from the order Artiodactyla, including Bovines (cows) and S wines (pigs) or of the order Perssodactyla, including Equines (horses). In some instances, the mammals are of the order Primates, Ceboids, or Simoids (monkeys) or of the order Anthropoids (humans and apes). In particular embodiments, the mammal is a human.

The following examples are given merely to illustrate the present invention and not in any way to limit its scope.

### EXAMPLES

### Example 1

### Optimization of Digestion Steps

A standard protocol for digesting adipose tissue for the release of cells from the tissue was optimized with regard to enzyme concentration and digestion time. The two parameters and overall digestion technique was optimized for obtaining the maximum amount of cells released per ml of adipose tissue, while maintaining cell viability.

Collagenase concentrations of 191 U/ml, 250 U/ml, and 319 U/ml were tested. The collagenase solutions which were used at a 1:1 volume ratio with the volume of adipose tissue (lipoaspirate) comprised the following components: Collagenase Type I (Worthington Biochemical); DMEM/F12 50:50 (Gibco); w/ L-Glutamine; 10% FBS (Hyclone); and 10 mM Hepes.

The times of incubation with the collagenase solutions were varied between 15 and 60 minutes. The digestions were carried out at 37 degrees C. with constant shaking. A wash buffer comprising DMEM (Gibco) and 10% FBS (Hyclone) also was used in these experiments to inactivate the enzyme to promote the overall health and viability of the released cells.

In addition to the total number of cells released from the adipose tissue, the viability of the released cells was measured. Cell viability was assayed with a dye (7AAD) that only permeates the cell when its membrane is compromised. The dye, and thus cell viability, was then quantified using flow cytometry.

The total number of cells released from adipose tissue, as well as the percentage of those cells that were viable, upon incubation with medium containing different concentrations of collagenase are shown in Table 1.

TABLE 1

| Concentration | Total Cells | | |
|---|---|---|---|
| (U/ml) | Released | Stdev | % Viability |
| 191 (n = 12) | 4.41E + 05 | 2.81E + 05 | 79.86 |
| 250 (n = 6) | 8.10E + 05 | 8.63E + 04 | 85.87 |
| 319 (n = 6) | 4.15E + 05 | 3.64E + 04 | 76.09 |

Total Cells Released represents total number of cells released per mL of adipose tissue.

These data indicated that incubation with a medium containing collagenase at a concentration of 250 U/mL for a time of 45 minutes at approximately 37 degrees C. with agitation, shaking, or mixing was optimal for this application. The number of cells released per ml of tissue processed under these conditions was greatest at 250 U/ml, and the cells' viability was not sacrificed to a large extent.

Optimization of Selection Steps

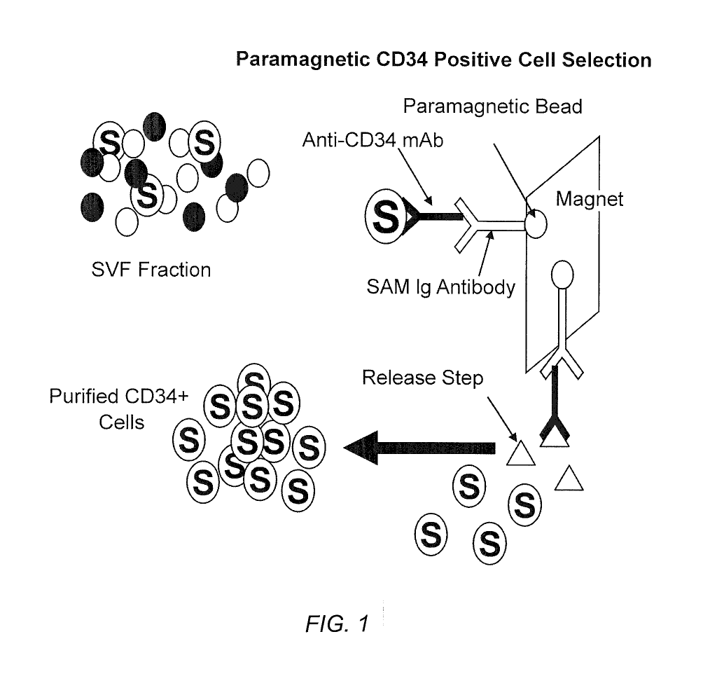

The cells released from adipose tissue upon collagenase digestion were enriched or selected for a target cell population using an antibody-based capture methodology. The selecting steps which are outlined in FIG. 1 were optimized for achieving the greatest amount of purity and % recovery, while maintaining cell viability.

Antibody concentrations, bead to target cell ratios, release peptide concentrations, and trituration techniques were analyzed by selecting for CD34-positive cells contained within the population of adipose tissue-released cells. CD34 expression and quantification of selected and unselected cells were assayed using a Stem Kit CD34 Enumeration Assay which calculates the total number of CD34-positive cells in a given sample using fluorospheres. CD45 expression and cell viability (via 7AAD) was additionally evaluated.

The solutions used for these experiments included a Selection Buffer and components of a Reagent Kit (Baxter). The Selection Buffer comprised Phosphate Buffered Saline (PBS) (Baxter); 137 mM NaCI; 2.68 mM KCl; 3.21 mM Na2HPO4×12 H2O (pH 7.2); 0.41% Sodium Citrate (Baxter); and 1% Human Serum Albumin (Baxter). The Reagent Kit (Baxter) comprised Anti-CD34 antibody (Clone9C5); Dynal anti Mouse paramagnetic beads (SAM); and PR34 release peptide.

Antibody concentrations (amount of antibody per million target cells) which mimicked Isolex kit concentrations and which were believed to be in excess were used. Using 1 μg of antibody per million target cell and optimizing for the specific type of cells used determined the concentration of the anti-CD34 antibody. Specifically, antibody concentrations between about 1 and 2.5 μg per million target cells were tested. The media containing the released cells and the antibody were incubated on a rotator for 30 minutes. Cells were washed by centrifugation at 600 g.

Bead to cell ratios are a critical aspect of selection methods and may vary based on the type of cells/tissue being processed. For this assay, which involved adherent cells expressing CD34 antigen at a higher frequency than peripheral blood derived cells, bead to target cell ratios of 1:1, 2:1, 4:1, 5:1, 7.5:1, and 10:1 were tested. The target cell was approximated at 50% of the total population of unselected, released cells. Beads were incubated with the cells on a rotator for about 45 minutes. The mixture was exposed to a magnet three times to capture the immunecomplexes. The target range of 1:1 to 5:1 (bead to target cell ratio) was determined to be optimal. This range may be optimal for adipose tissue only.

While other bead to target cell ratios appeared to be useful, allowing for extremely high capture efficiencies, release efficiency was believed to suffer at too high bead to target cell ratios.

Optimal release peptide concentrations were determined by varying the amount of PR34 peptide at 1 mg/ml or 2 mg/ml. The medium containing the cells, CD34 antibody, beads and release peptide were incubated on a rotator for 45 to 60 minutes.

The adherent nature of the CD34-positive target cells required a method of dispersing clumps of cells, as well as creating a space for release peptide binding. Trituration was used to break the clumps and release cells during the release step. Specifically, the clumps were triturated by drawing the medium into a small bore of a syringe and slowly expelled out of the syringe at three separate times during the release step. Trituration times tested included 0, 15, 30, and 45 minutes.

From these experiments, it was found that use of the release peptide, PR34, with trituration yielded the best results. This type of trituration technique is specific and novel to adipose tissue. An approximate release percentage greater than 20% was observed using this technique.

The viability and purity of the selected CD34-positive cells were compared to that of unselected cells. As shown in Table 2, the antibody-based selection steps achieved an approximate 2-fold increase in purity (from 40% to 75%) with a total % recovery of 23%. In another set of experiments, the selection steps achieved an increase purity of CD34-positive cells from 36% to 73% (data not shown). Notably, the selection steps did not significantly comprise the viability of the cells.

TABLE 2

| | | | | | % Recovery |
|---|---|---|---|---|---|
| | Viability | | Purity | | (Selected |
| | Unselected | Selected | Unselected | Selected | Population) |
| Average | 83.32 | 74.78 | 40.26 | 75.11 | 23.59 |
| Stdev | 8.43 | 11.45 | 13.55 | 7.71 | 5.42 |

For this example, n = 3.
Purity is a measure of the total number of CD34-positive cells divided by the total cells (excluding debris).
% recover is the total number of CD34-positive cells in the selected population divided by the total number of CD34-positive cells in the unselected population.

The phenotypes of the selected cells were determined to be different from unselected cells. For example, CD45 expression was decreased in the CD34 selected cells (7.54%) as compared to the unselected cells (15.82%). Also, the lymphocytes also were decreased upon selection. Also, the number of bright CD34 cells (cells which express CD34 to a higher degree) was decreased in the selected cell population as compared to the unselected population. The selection steps can be tailored to capture the bright CD34 cells. The number of CD140b-positive cells increased upon selection, while the number of ASCs and CD144-negative cells were maintained upon selection.

### Example 2

Human adipose tissue from a liposuction procedure was digested using the optimal enzyme digestion procedure as determined in Example 1. Specifically, a solution comprising 250 U/ml media was added to equal volume of adipose tissue. The mixture was incubated at 37 degrees C. with continuous shaking. The enzyme was inactivated with a high concentration fetal bovine serum (FBS) solution. The inactivated enzyme mixtures was centrifuged at 300 g for 5 minutes and the cell pellet was resuspended in a new solution. The cell suspension was subjected to a series of filtration steps. The filtered solution was then centrifuged at 300 g for 5 min. The cell pellet was resuspended in a new solution. This cell product was referred to as the stromal vascular fraction (SVF).

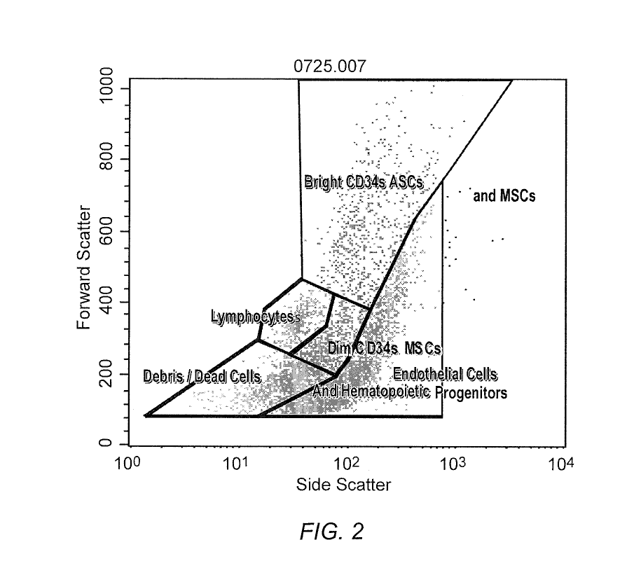

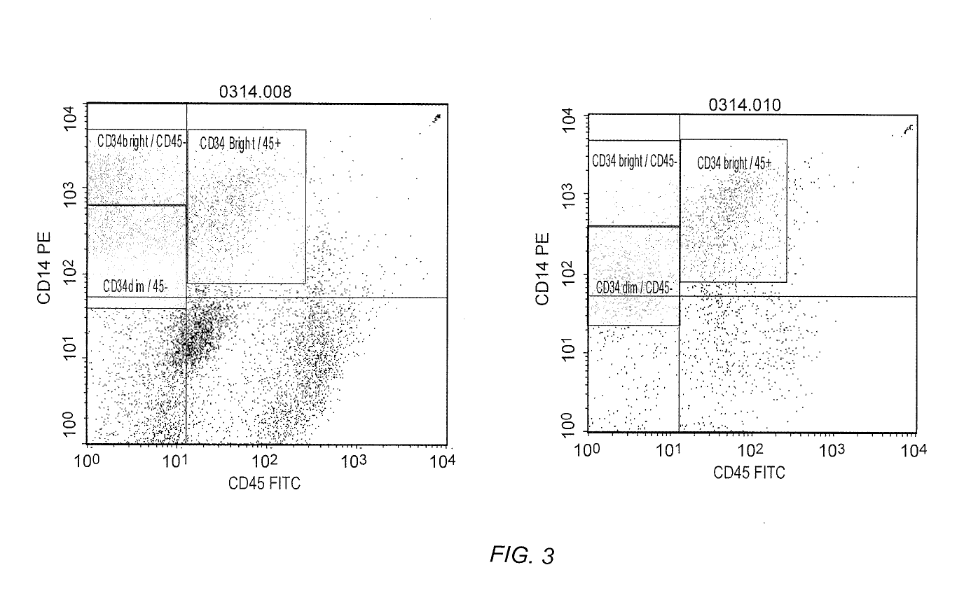

The SVF samples were analyzed on FACSCalibur and FACScan Flow Cytometers (Becton Dickinson, San Jose, Calif.). A multi-color flow cytometric panel was used to quantitatively determine the cellular composition of the SVF (FIG. 2). The presence of bright CD34-positive cells (adipose stromal cells; ASCs), lymphocytes, dim CD34-positive cells (mesenchymal stromal cells; MSCs), endothelial cells and debris (dead cells) was determined and quantitated (FIG. 3).

Targeted selection of the SVF for CD34-positive cells was performed using CD34 antibody and paramagnetic beads. The cells were rosetted upon incubation with paramagnetic beads displaying a secondary antibody directed to the CD34-specific antibody. The CD34-positive cells were released from the antibody-bead complex via a release step. Purified CD34-positive cells were thus obtained by this selection procedure.

Flow cytometry was used to determine the phenotype of the CD34-positive cells. Expression of the cell markers including CD10, CD13, CD34, CD45, CD140b, CD90, CD31, CD105, CD73, CD144, and others was determined in this manner. CD34 is a stem cell marker which is present on many cell types including stem cells of any cell source, blast cells and various cells in the bone marrow and umbilical cord. CD140b (also known as PDGFR2) has been reported as a MSC marker. CD90 is also known as Thy-1 Thymus Cell Antigen and is present on many cell types including but not limited to MSCs, HSCs, NK cells, endothelium, and fibroblasts. CD31 is also known as PECAM-1 (Platelet Endothelial Cellular Adhesion Molecule) and is present on Endothelial Cells. CD105 (also known as Endoglin, a regulatory component of the TGF-beta receptor-cell complex) mediates cellular response to TGF-beta. CD105 is present on many cells, as it helps regulate proliferation and apoptosis pathway. CD73 is a Ecto-5′-nucleotidase (5′-NT) and is present on leucocytes but has been reported as an ASC marker. CD45 is a leucocyte common antigen and is present on all hematopoietic cells except erythrocytes. CD144 is also known as VE-Cadherin, a calcium dependent adhesion molecule at the intercellular junctions, and is found mainly on vascular endothelium. Recent research indicates that CD 144 may also be present on some leucocytes as well.

Within the population of CD34-positive selected cells of the unselected population, at least three distinct sub populations appear to exist: Population A comprises 20+/−6% of the total and has an expression profile of 34 bright/45−/90+/140b+/31−/73+/44+/105−/146−; Population B comprises 8+/−4% of the total and has an expression profile of 34 bright/45−/90+/140b−/105+/146+/144+/31+/44+; and Population C comprises 31+1−16% of the total and has an expression profile of 34 dim/45−/90+/105+/146+/31+/44 dim. About 96% of Populations A and B are CD34-positive and about 78% of these populations are positive for CD140b expression. About 92% of these populations are negative for CD45 expression.

Modifications to the selection method resulted in the retention or elimination of these cell populations.

Population A appeared to be adipose stromal cells of a multipotent nature. These cells display the stem cell marker and the profile of 45−/105+/31−, which implies that they cells have stromal properties. The stromal properties of this population may allow these cells to have tissue engineering qualities. These cells also express the PDGFR2 antigen, which implies a more mesenchymal nature. The combination of these markers may allow these cells to differentiate into osteocyte, chondrocyte, adipocyte, myocyte, epithelial cell, neuron or others.

Population B had an endothelial profile (31+/144+/146+) but also displays the stem cell marker and the endoglin marker CD105, part of the TGFB receptor. These cells may facilitate angiogenesis and vasculogenesis as well as contribute to tissue repair.

Population C displayed the profile 34+/31+/146+/105 dim/90+/45−, which has been published as a profile of hematopoietic progenitors, capable of forming myeloid as well as lymphoid cells. Other markers suggest there may be endothelial properties of these cells as well.

The SVF is a cell fraction rich in therapeutic potential. Each population contains specific characteristics reflected in its surface marker profile. A selection method for each of these populations could be designed to isolate one population without contamination from the others. These specific cell products would have multiple specific therapeutic uses and could be specialized to the needs of individual patients.

### Example 3

Fresh ASCs were prepared from human lipoaspirate. After collagenase treatment and filtration through 100 micron filter, the sample was split: ¾ used for selecting a CD34-positive fraction with Dynabeads (Invitrogen), as essentially described in Examples 1 and 2, and ¼ processed by a standard filtration method. CD34-selection achieved an approximate 3-fold enrichment of CD34-positive cells, increasing the purity of CD34-positive cells from 13% to 36%.

A hindlimb ischemia model was created in nude mice. One day after surgery, animals were assigned to 1 of 3 groups: PBS control; Unselected (total population); and CD34-selected. The treatments (PBS, unselected cells, or CD34-selected cells) were administered by direct intramuscular injection into the gastrocnemius and quadriceps muscles of ischemic limbs.

Laser Doppler perfusion imaging (LDI) was performed at days 1, 5, 10, 15 and 20 to assess reperfusion of the ischemic limb. The majority of control-treated mice with the lowest relative perfusion at day 1 developed severe necrosis (affecting more than half of the ischemic foot) by day 5 and had to be euthanized. Consequently, the mean value for the control group was skewed towards higher relative perfusion. No animals were excluded based on out of range day 1 relative perfusion values (i.e., >20%) since this would have required removing 3 of the remaining 5 control mice.

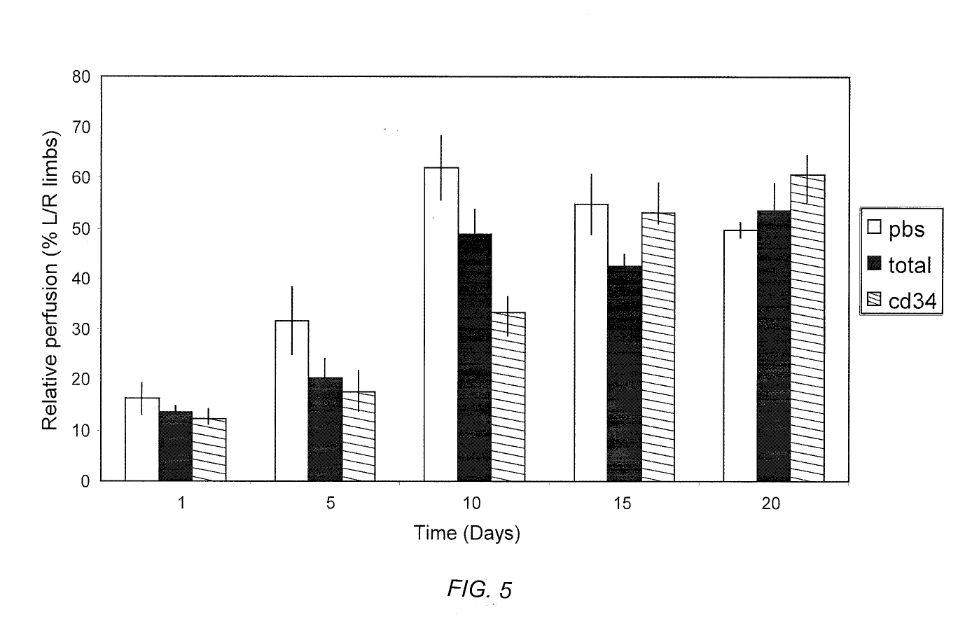

As shown in FIG. 5, a high relative perfusion value was observed at days 5 and 10. This observation may be explained by a higher number of exclusions of control group mice.

A 3-fold enrichment of CD34+ ASCs was more potent than unselected cells when injected directly into the affected musculature of mice with ischemic hindlimbs.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Certain embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of obtaining a population of cells from adipose tissue, comprising incubating the adipose tissue in a solution comprising an enzyme at a concentration which is at least about 200 U/ml solution and not more than about 319 U/ml solution for a time period between about 30 and about 60 minutes, thereby obtaining a population of cells from the adipose tissue.

2. The method of claim 1, wherein the enzyme concentration is between about 200 and about 300 U/ml solution, between about 225 and about 275 U/ml solution, or between about 245 and 255 U/ml solution.

3. (canceled)

4. (canceled)

5. The method of claim 1, wherein the time period is between about 45 to about 55 minutes.

6. The method of claim 1, wherein the enzyme is collagenase, trypsin, dispase, or a mixture of enzymes comprising at least one of the foregoing.

7. The method of claim 1, wherein the ratio of adipose tissue volume to enzyme solution volume is about 1:1.

8. The method of claim 1, wherein at least 6.0×10⁵ cells or at least 8.0×10⁵ cells per ml adipose tissue are obtained.

9. (canceled)

10. A method of obtaining an enriched population of target cells from adipose tissue, comprising:
a. obtaining a population of cells from adipose tissue by incubating adipose tissue in a solution comprising an enzyme at a concentration which is at least about 200 U/ml solution and not more than about 319 U/ml solution for a time period between about 30 and about 60 minutes; and
b. incubating the population of cells obtained in (a) with a primary antibody that separates the population of cells into a subpopulation comprising target cells and a subpopulation substantially devoid of target cells, thereby obtaining an enriched population of target cells.

11.-14. (canceled)

15. The method of claim 10, wherein the target cells are CD34-positive cells.

16. The method of claim 10, comprising incubating the population of cells with beads, thereby forming complexes comprising a bead and the primary antibody.

17. (canceled)

18. (canceled)

19. The method of claim 16, wherein the beads are present at a bead to target cell ratio between 1:1 and 5:1, or wherein the bead to target cell ratio is about 4.1.

20. (canceled)

21. The method of claim 16, comprising removing the complexes from the population of cells.

22. The method of claim 21, wherein the beads are paramagnetic beads and the complexes are removed from the population of cells with a magnet.

23. The method of claim 16, comprising incubating the complexes with a release peptide.

24. The method of claim 23, wherein the release peptide comprises an epitope of CD34 or an epitope of the primary antibody.

25. The method of claim 23, wherein the release peptide is a soluble fragment of CD34 or a PR34 release peptide.

26. The method of claim 23, further comprising triturating the complexes.

27. The method of claim 1, (i) wherein the population obtained is cultured or plated for less than one day, (ii) which is devoid of any steps which expand the population obtained, or (iii) both (i) and (ii).

28. The method of claim 10, (i) wherein the population obtained is cultured or plated for less than one day, (ii) which is devoid of any steps which expand the population obtained, or (iii) both (i) and (ii).

29. An enriched population of target cells obtained from adipose tissue according to the method of claim 10.

30. (canceled)

31. (canceled)

32. A method of preparing a pharmaceutical composition comprising cells for administration to a patient, comprising formulating the enriched population of target cells of claim 29 with a pharmaceutically acceptable carrier.

33.-35. (canceled)

36. A pharmaceutical composition prepared according to the method of claim 32.

37. A method of treating a disease or medical condition in a patient, comprising administering to the patient the pharmaceutical composition of claim 36 in an amount effective to treat the disease or medical condition.
